(19) United States
(12) Patent Application Publication
(10) Pub. No.: US 2014/0031770 A1
(43) Pub. Date: Jan. 30, 2014

(54) TRANSDERMAL PATCH

(71) Applicant: Hsiu-Wei Lin, Tainan City (TW)

(72) Inventor: Hsiu-Wei Lin, Tainan City (TW)

(21) Appl. No.: 13/557,233

(22) Filed: Jul. 25, 2012

(51) Int. Cl. A61M 35/00

(52) U.S. Cl. 604/307

(57) ABSTRACT

The transdermal patch contains a base layer, a textured pharmaceutical layer, and a detachable layer, sequentially stacked from top to bottom. The textured pharmaceutical layer contains pressure-sensitive adhesive and medicament, and is configured with at least an embossed pattern for contacting with the skin. After the detachable layer is peeled off from the textured pharmaceutical layer and the latter is attached to the skin, the transfer performance of the contained medicament is significantly enhanced by the embossed pattern's increased area of release to the medicament.

### TECHNICAL FIELD OF THE INVENTION

The present invention generally relates to transdermal patches, and especially relates to a transdermal patch having increased area of release for the transdermal patch's medicament.

### DESCRIPTION OF THE PRIOR ART

A conventional transdermal patch mainly contains a base layer, a pharmaceutical layer, and a detachable layer. The pharmaceutical layer contains pressure-sensitive adhesive and medicament. To apply the transdermal patch, the detachable layer is first peeled off and the pharmaceutical layer is attached to the skin.

A common issue for various transdermal patches is the limited transfer performance of the contained medicament through the flat contact with the skin.

### SUMMARY OF THE INVENTION

Therefore, a novel transdermal patch is provided which contains a base layer, a textured pharmaceutical layer, and a detachable layer, sequentially stacked from top to bottom. The textured pharmaceutical layer contains pressure-sensitive adhesive and medicament, and is configured with at least an embossed pattern for contacting with the skin.

After the detachable layer is peeled off from the textured pharmaceutical layer and the latter is attached to the skin, the transfer performance of the contained medicament is significantly enhanced by the embossed pattern's increased area of release to the medicament.

The foregoing objectives and summary provide only a brief introduction to the present invention. To fully appreciate these and other objects of the present invention as well as the invention itself; all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a sectional diagram showing a transdermal patch according to a first embodiment of the present invention.

FIG. 2 is a sectional break-down diagram with enlarged views to transdermal patch of FIG. 1.

FIG. 2A is an enlarged view of a portion of FIG. 2.

FIG. 2B is an enlarged view of a portion of FIG. 2.

FIG. 3 shows an application scenario to a transdermal patch of the present invention implemented as a face mask.

FIG. 4 is a sectional diagram showing a transdermal patch according to a second embodiment of the present invention.

FIG. 5 is sectional diagram showing the transdermal patch of FIG. 4 after it is applied to skin.

FIG. 6 is a perspective diagram showing that an embossed pattern is contained in the transdermal patch of FIG. 4.

FIG. 7 is a sectional diagram showing a transdermal patch according to a third embodiment of the present invention.

FIG. 8 is sectional diagram showing the transdermal patch of FIG. 7 after it is applied to skin.

FIG. 9 is a perspective diagram showing that a graphic pattern is manifested from the transdermal patch of FIG. 7.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

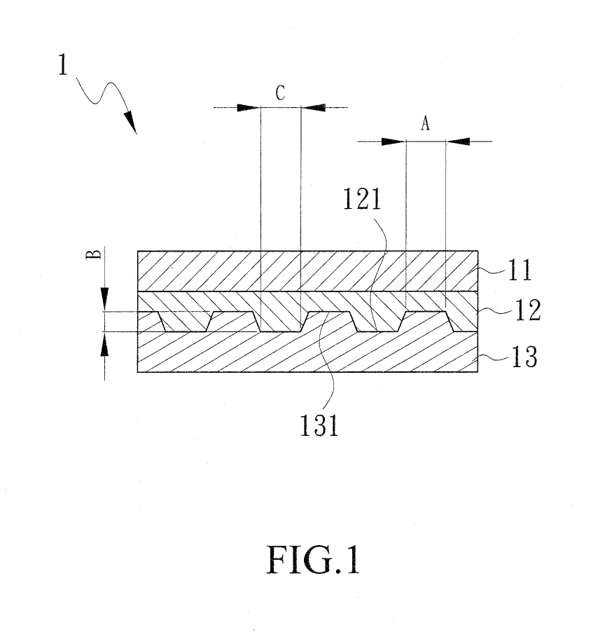

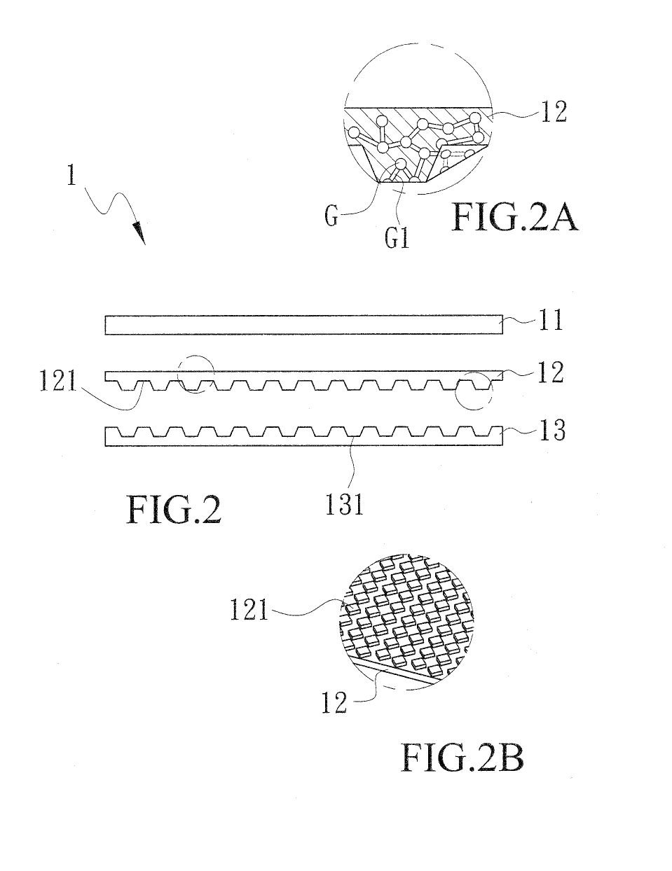

As shown in FIGS. 1 and 2, a transdermal patch 1 according to an embodiment of the present invention contains a base layer 11, a textured pharmaceutical layer 12, and a detachable layer 13, sequentially stacked from top to bottom.

The base layer 11 is an airproof film made of an environmentally friendly material such as PP, PE, PEVA, PET, etc.

The textured pharmaceutical layer 12 contains pressure-sensitive adhesive and medicament, and is adhered to the base layer 11 with an appropriate thickness relative to the base layer 11. The textured pharmaceutical layer 12 has at least a first embossed pattern 121 for contacting with the skin. As shown in FIGS. 2, 2A and 2B, numerous cavities G are configured in the textured pharmaceutical layer 12 and the cavities G are connected with channels G1. These cavities G are similar to the cavities of a cork and they provide the storage for medicament such as those for skin treatment, plant abstracts, drugs, etc. The details about the medicament should be familiar to those of the related arts and are omitted here as it is not the subject matter of the present invention.

The detachable layer 13 is attached to a bottom side of the textured pharmaceutical layer 12. As shown in FIGS. 2, 2A and 2B, the detachable layer 13 is pressed to form a second embossed pattern 131 that compliments the first embossed pattern 121. After the detachable layer 13 is peeled off and the first embossed pattern 121 is exposed and attached to the skin, the first embossed pattern 121 does not entirely contact with the skin and as such provides a greater area of release for the medicament.

As shown in FIG. 5 where the transdermal patch 1 has its detachable layer 13 removed and is applied to the skin 14, the transdermal patch l′s textured pharmaceutical layer 12 forms incomplete contact with the skin 14 due to the first embossed layer 121, and the medicament is released, as indicated by the arrow heads, through a greater area size.

More specifically, the first embossed pattern 121 contains a large number of peaks and troughs. The medicament before release is stored in the numerous cavities and channels G and G1, and every peak or trough in the first embossed pattern 121 has three neighboring faces, thereby constituting a greater area of release. The medicament stored in the cavities and channels G and G1 permeates through the greater area of release and conducts ion exchange with the skin 14 (as indicated by the arrow heads). The medicament as such would be quickly absorbed by the skin 14 and takes effect at a greater speed. Preferably, the width of each peak is between 0.3 and 0.8 mm; the width of each trough is between 0.1 and 0.2 mm; and the distance between each pair of neighboring peak and trough is between 0.02 to 0.05 mm.

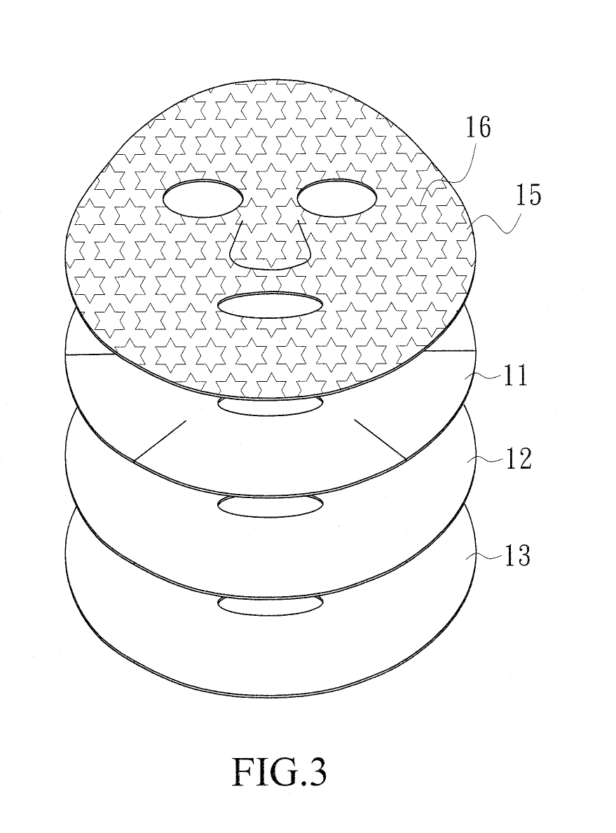

FIG. 3 depicts an application scenario of the transdermal patch 1 where it is packaged as a face mask. To add the appeal of the transdermal patch 1, a first graphic layer 16 is coated on a top side of a graphic support layer 15, which in turn is attached to a top side of the base layer 11. The graphic support layer 15 is made of acrylic o PU. The first graphic layer 16 in the present embodiment manifests a large number of star shapes.

The purpose of the first graphic layer 16 is to present a visual effect for the transdermal patch 1. Various texts, graphics, or images can be ink-printed on the graphic support layer 15. A user can even paint the first graphic layer 16, therefore making the transdermal patch 1 more interesting in its usage.

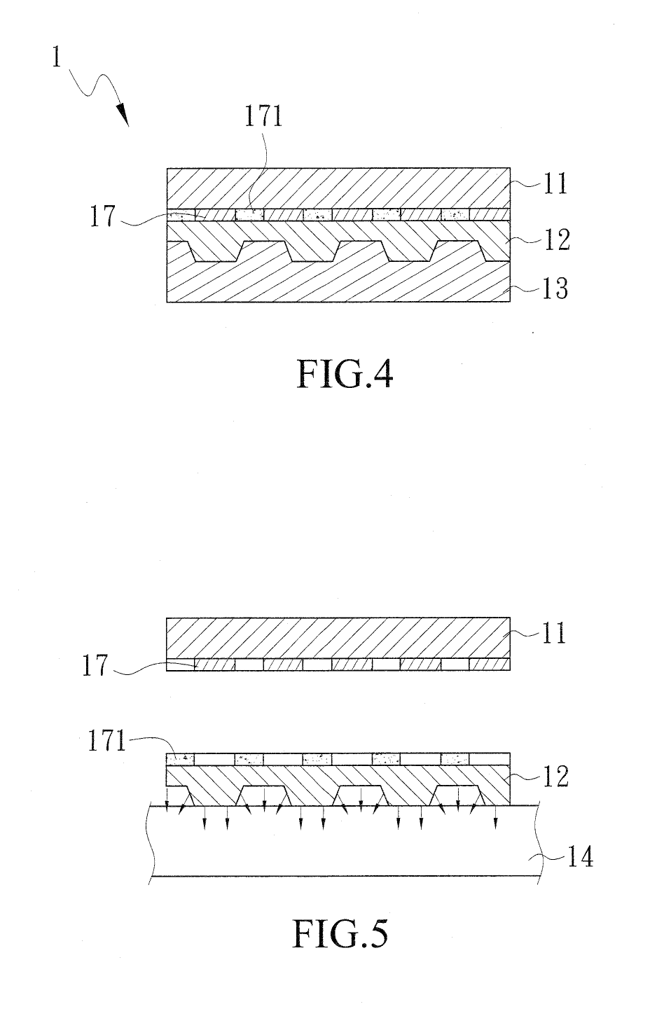

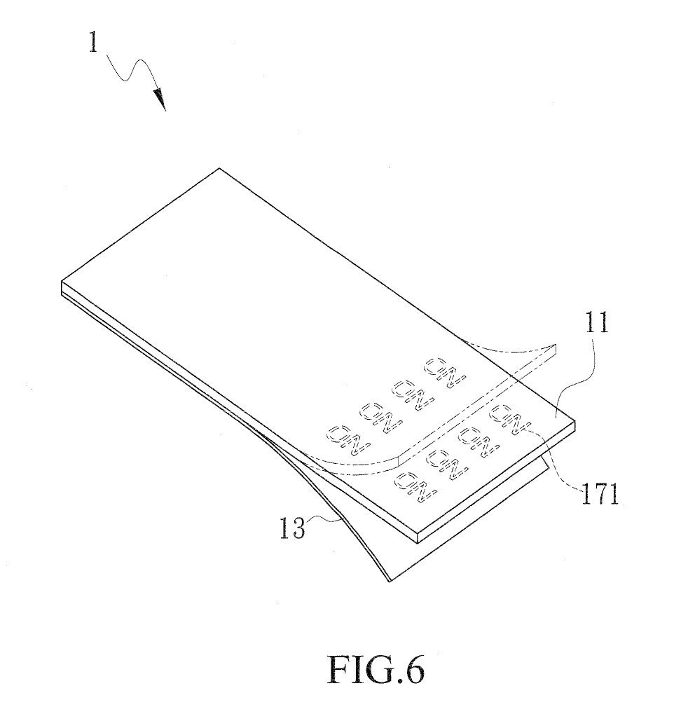

FIGS. 4 and 5 depict a transdermal patch 1 according to a second embodiment of the present invention. FIG. 4 shows the transdermal patch 1 when all layers are present. FIG. 5 shows the transdermal patch 1 when the detachable layer 13 is removed. In the present embodiment, a partially detachable layer 17, also having a graphic pattern, is sandwiched between the base layer 11 and the textured pharmaceutical layer 12. The partially detachable layer 17 allows the base layer 11 to be peeled off and, when that happens, due to the adhesives of different strengths applied to the graphic pattern of the partially detachable layer 17, a second embossed pattern 171 complementing the graphic pattern is left on the textured pharmaceutical layer 12. In the present embodiment, as shown in FIG. 6, the second embossed pattern 171 shows a number of texts “ON,” indicating that the transdermal patch 1 is in use.

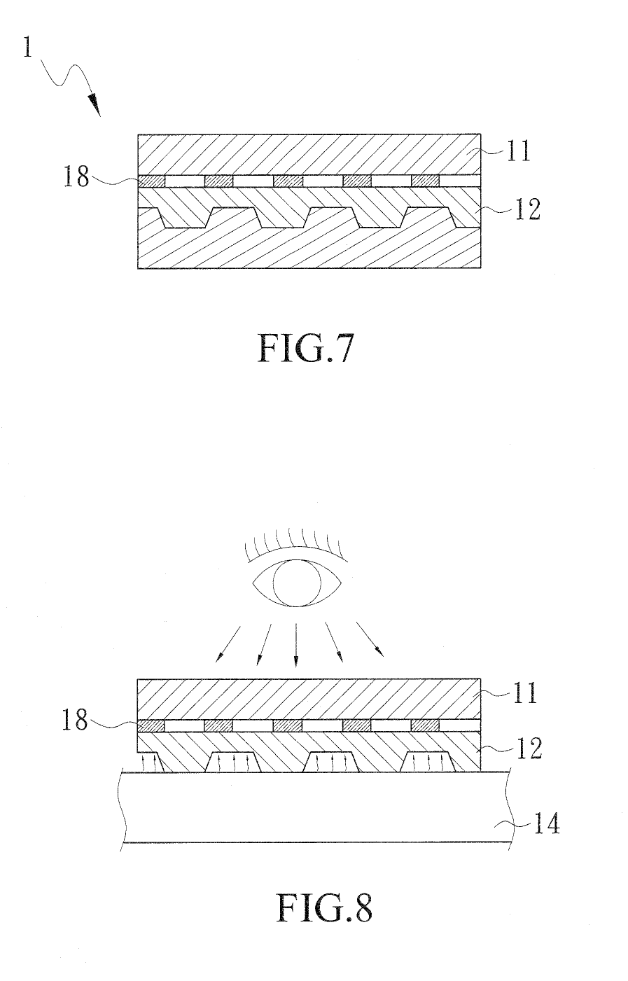

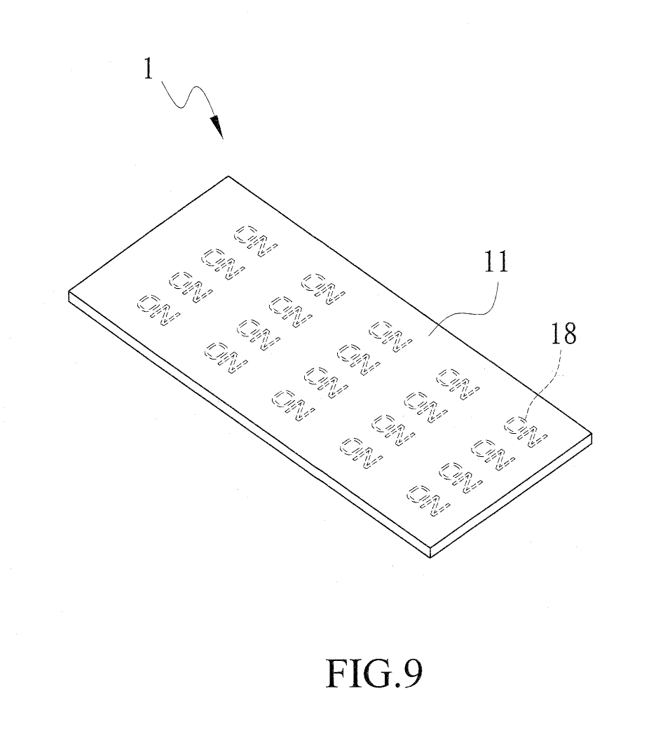

FIGS. 7 and 8 are illustrations of a transdermal patch 1 according to a third embodiment of the present invention. FIG. 7 shows the transdermal patch 1 when the detachable layer is not removed and FIG. 8 shows the transdermal patch 1 is applied to skin 14 after the detachable layer 13 is removed. In the present embodiment, a second graphic layer 18 is sandwiched between the base layer 11 and the textured pharmaceutical layer 12, and the base layer 11 would become transparent when it is moisturized. After the textured pharmaceutical layer 12 is attached to the skin 14 for a period of time, the vapor produced from the skin 14 would permeate through the porous textured pharmaceutical layer 12 and the second graphic layer 18 into the base layer 11. As such, the texts, graphics, or images of the second graphic layer 18 manifest through the moisturized and transparent base layer 11. In the present embodiment, as shown in FIG. 9, the second graphic layer 18 shows a number of texts “ON,” indicating that the transdermal patch 1 is in use.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

1. A transdermal patch, comprising a base layer, a textured pharmaceutical layer, and a detachable layer, sequentially stacked from top to bottom; wherein
the textured pharmaceutical layer contains pressure-sensitive adhesive, at least a first embossed pattern for contacting with skin; a plurality of internal cavities connected with channels in the textured pharmaceutical layer; and medicament stored in the cavities and channels;
the first embossed pattern contains a plurality of peaks and troughs so as to provide a greater area of release for the medicament;
the detachable layer has a second embossed pattern that compliments the first embossed pattern;
the width of each peak is between 0.3 and 0.8 mm;
the width of each trough is between 0.1 and 0.2 mm; and
the distance between each pair of neighboring peak and trough is between 0.02 to 0.05 mm.

2. (canceled)

3. The transdermal patch according to claim 1, further comprising a graphic support layer on a top side of the base layer; and
a first graphic layer coated on a top side of the graphic support layer.

4. The transdermal patch according to claim 1, further comprising:
a partially detachable layer sandwiched between the base layer and the textured pharmaceutical layer;
wherein the partially detachable layer has a graphic pattern; the partially detachable layer allows the base layer to be peeled off; and, after the base layer and the partially detachable layer are peeled off, a third embossed pattern complementing the graphic pattern is left on the textured pharmaceutical layer.

5. The transdermal patch according to claim 1, further comprising:
a second graphic layer sandwiched between the base layer and the textured pharmaceutical layer;
wherein the second graphic layer has a graphic pattern; the base layer becomes transparent when it is moisturized; and, after the textured pharmaceutical layer is attached to skin, the graphic pattern of the second graphic layer manifests through the moisturized and transparent base layer.
